(19) United States
(12) Patent Application Publication
(10) Pub. No.: US 2019/0365457 A1
(43) Pub. Date: Dec. 5, 2019

(54) BIPOLAR FORCEPS FOR HEPATIC TRANSECTION

(71) Applicant: COVIDIEN LP, Mansfield, MA (US)

(72) Inventors: DANIEL A. JOSEPH, GOLDEN, CO (US); JOHN A. HAMMERLAND, III, ARVADA, CO (US)

(21) Appl. No.: 16/392,980

(22) Filed: Apr. 24, 2019

Related U.S. Application Data
(60) Provisional application No. 62/677,806, filed on May 30, 2018.

(51) Int. Cl. A61B 18/14; A61B 18/12

(52) U.S. Cl. CPC A61B 18/1445; A61B 2018/00529; A61B 2018/126; A61B 18/1206

(57) ABSTRACT

A forceps includes first and second shafts configured to support an end effector assembly at a distal end thereof. The end effector includes first and second opposing jaw members each having an electrically conductive plate associated therewith configured to communicate electrosurgical energy between electrically conductive plates upon selective activation thereof. The first and second jaw members pivotable relative to one another about a pivot such that the jaw members are selectively movable between an open position wherein the jaw members are spaced relative to one another and a closed position for grasping tissue therebetween. A fluid line is integrated with one of the shafts and is configured to selectively deliver fluid from a fluid source to the end effector assembly proximate the electrically conductive plates during electrical activation.

### CROSS REFERENCE TO RELATED APPLICATION

The present application claims the benefit of and priority to U.S. Provisional Application Ser. No. 62/677,806, filed on May 30, 2018 the entire contents of which are incorporated herein by reference.

### BACKGROUND

### Technical Field

The present disclosure relates to surgical instruments and, more particularly, to an open surgical forceps for use with hepatic surgical transection.

### Description of Related Art

Hepatic resection is a surgical procedure with many challenges due to an increased risk of bleeding and complications relating to the anatomy of the liver, i.e., complexity of the biliary and vascular anatomy of the liver. Liver transection is the most challenging part of hepatic resection and is associated with a risk of possible hemorrhage. Understanding the segmental anatomy of the liver and delineation of the proper transection plane using intraoperative ultrasound are prerequisites to safe liver transection. The most important factor for a better outcome is reduced blood loss due to improvements in surgical instruments and with surgical techniques.

Various surgical techniques have been used in the past to facilitate liver transection, so-called clamp crushing and the use of intraoperative ultrasound being the most prominent. More recently, technological advances have led to the development of new instruments for use with liver transections, e.g., ultrasonic dissectors, so-called “water jet” instruments, and commercially named instruments sold under the tradenames Harmonic Scalpel, Ligasure®, and TissueLink, for example. Moreover, advances in operative techniques have also contributed to a reduction in blood loss during liver transection. These include better delineation of the transection plane with the use of intraoperative ultrasound, and better inflow and outflow control of fluids.

These new instruments utilize various types of energy modalities to coagulate or seal vessels. These include ultrasonic and radiofrequency devices or the above-mentioned commercially available instruments sold under the names Harmonic Scalpel, Ligasure® and TissueLink. These new instruments may be used alone or in combination with clamp crushing or ultrasonic dissection to improve the safety of liver transection. With this technology, the liver parenchyma tissue is fragmented with ultrasonic energy and aspirated, thus exposing vascular and ductal structures that can be ligated or clipped with titanium hemoclips.

Ligasure® (Valley Lab, Tyco Healthcare (now Medtronic), Boulder, Colo., USA) is another device designed to seal small vessels using a different principle. By a combination of compression pressure and bipolar radiofrequency (RF) energy, the various instruments cause shrinkage of collagen and elastin in the vessel wall, and these instrument are effective in sealing small vessels up to 7 mm in diameter. Ligasure® in combination with a clamp crushing technique has resulted in lower blood loss and faster transection speed in minor hepatic resections compared with conventional techniques of electric cautery or ligature for controlling vessels in the transection plane.

Ultrasonic shear (Harmonic Scalpel, Ethicon Endo-Surgery, Cincinnati, Ohio, USA), uses ultrasonically activated shears to treat small vessels between the vibrating blades The blade's longitudinal vibration dissects liver parenchyma. A coagulation effect is caused by protein denaturation, which occurs as a result of destruction of the hydrogen bonds in proteins and generation of heat in the vibrating tissue. A tissue-cutting effect derives from a saw mechanism in the direction of the vibrating blade. While the benefit of the use of Harmonic Scalpel in open hepatic resection remains uncertain, it is commonly used in laparoscopic hepatic resection, especially for resection of peripheral lesions.

RF ablation (RFA) is a relatively new technique for liver transection. A Cool-tip® RF electrode (sold by Medtronic, Inc.) is inserted along the transection plane and RF energy is applied to create overlapping cylinders of coagulated tissue, followed by transection of the coagulated liver using a simple scalpel. This device and technique has the advantage of simplicity compared with the aforementioned transection devices and techniques but tends to sacrifice too much parenchymal tissue.

### SUMMARY

As used herein, the term “distal” refers to the portion that is being described which is further from a user, while the term “proximal” refers to the portion that is being described which is closer to a user.

In accordance with one aspect of the present disclosure, a forceps includes first and second shafts configured to support an end effector assembly at a distal end thereof. The end effector includes first and second opposing jaw members each including an electrically conductive plate associated therewith configured to communicate electrosurgical energy therebetween upon selective activation thereof. One or both of the first and second jaw members is pivotable relative to the other about a pivot such that the jaw members are selectively movable between an open position wherein the jaw members are spaced relative to one another and a closed position for grasping tissue therebetween. A fluid line is integrated with one or both the first and second shafts of the forceps and is configured to selectively deliver fluid from a fluid source to the end effector assembly proximate the electrically conductive plates during electrical activation.

In one aspect, a sensor is included that senses the presence of fluid between the electrically conductive plates prior to selective activation of electrical energy. In aspects, the fluid is delivered through a nozzle disposed at a proximal end of one of the electrically conductive plates.

In other aspects, a rate of delivery of the fluid relates to an amount energy delivered to the electrically conductive plates. The rate of delivery of the fluid may be proportional to the amount of energy being delivered to the electrically conductive plates. The rate of delivery of the fluid may be linearly related to the amount of energy being delivered to the electrically conductive plates.

In yet other aspects according to the present disclosure, the fluid is electrically conductive, e.g., saline. The fluid may also be electrically non-conductive.

In still other aspects, a spacer is selectively disposed between the first and second shafts and is configured to maintain the jaw members in the spaced position during electrical activation.

In accordance with another aspect of the present disclosure a method of treating tissue is disclosed and includes orienting a forceps having first and second jaw members including electrically conductive opposing plates so that an edge of each electrically conductive plate contacts tissue. The method also includes: opening the first and second jaw members to create an area therebetween; delivering fluid to the area defined between the first and second jaw members; energizing the electrically conductive plates; and moving the first and second jaw members across tissue to create a desired tissue effect.

In one aspect, the method includes moving the first and second jaw members in a paint brush-like manner across tissue. In other aspects, the fluid is delivered at a rate relative to an amount of energy being provided to the electrically conductive plates. The fluid may be delivered at a rate that is linear to an amount of energy being provided to the electrically conductive plates.

In still other aspects, a spacer is provided to maintain the area between the first and second jaw members.

### BRIEF DESCRIPTION OF THE DRAWINGS

Various aspects of the present disclosure are described herein with reference to the drawings wherein like reference numerals identify similar or identical elements:

FIG. 1 is a side, perspective view of a forceps including opposing shaft members and an end effector assembly disposed at a distal end thereof according to an aspect of the present disclosure;

FIG. 2 is side view of the forceps of FIG. 1 including an irrigation line mechanically coupled therewith and configured to dispense fluid to a treatment site;

FIG. 3 is an enlarged distal perspective view of the end effector assembly including first and second jaw members and a distal end of the irrigation line disposed therebetween; and

FIG. 4 is an enlarged view of the forceps of FIG. 1 shown treating tissue and having a jaw stop engaged between shaft members to help maintain the first and second jaw members in a spaced apart orientation during tissue treatment.

### DETAILED DESCRIPTION

Throughout the description, like reference numerals and letters indicate corresponding structure throughout the several views. Also, any particular feature(s) of a particular exemplary embodiment may be equally applied to any other exemplary embodiment(s) of this specification as suitable. In other words, features between the various exemplary embodiments described herein are interchangeable as suitable, and not exclusive.

Embodiments of the disclosure include systems, devices, and methods to control tissue temperature at a tissue treatment site during an electrosurgical procedure, as well as shrinking, coagulating, cutting, and sealing tissue against blood and other fluid loss, for example, by shrinking the lumens of blood vessels (e.g., arteries or veins). In some embodiments, the devices may be configured, due to the narrow electrode size, to fit through a trocar down to a size as small as 5 mm.

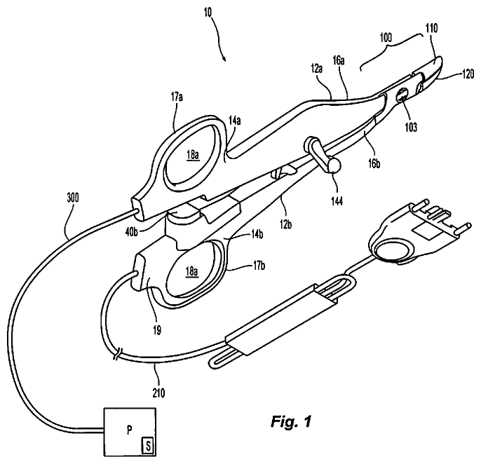

Referring now to FIG. 1, an open forceps 10 contemplated for use in connection with traditional open surgical procedures is shown. For the purposes herein, either an open instrument, e.g., forceps 10, or an endoscopic instrument (not shown) may be utilized in accordance with the present disclosure. Obviously, different electrical and mechanical connections and considerations apply to each particular type of instrument; however, the novel aspects with respect to the end effector assembly and its operating characteristics remain generally consistent with respect to both the open and endoscopic configurations.

With continued reference to FIG. 1, forceps 10 includes two elongated shafts 12a and 12b, each having a proximal end 14a and 14b, and a distal end 16a and 16b, respectively. Forceps 10 further includes an end effector assembly 100 attached to distal ends 16a and 16b of shafts 12a and 12b, respectively. End effector assembly 100 includes a pair of opposing jaw members 110 and 120 that are pivotably connected about a pivot 103. Each shaft 12a and 12b includes a handle 17a and 17b disposed at the proximal end 14a and 14b thereof. Each handle 17a and 17b defines a finger hole 18a and 18b therethrough for receiving a finger of the user. As can be appreciated, finger holes 18a and 18b facilitate movement of the shaft members 12a and 12b relative to one another between a spaced-apart position and an approximated position, which, in turn, pivot jaw members 110 and 120 from an open position, wherein the jaw members 110 and 120 are disposed in spaced-apart relation relative to one another, to a closed position, wherein the jaw members 110 and 120 cooperate to grasp tissue therebetween.

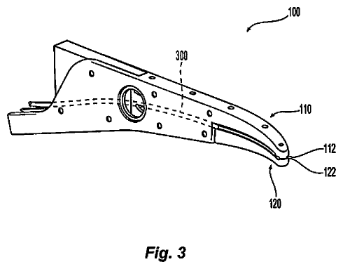

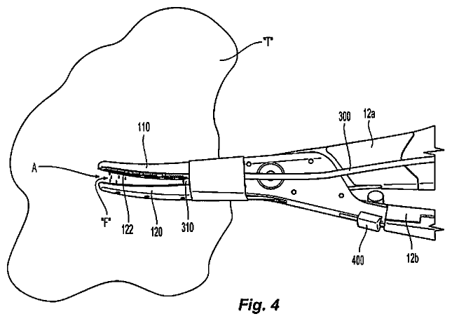

Continuing with reference to FIG. 1, one of the shafts, e.g., shaft 12b, includes a proximal shaft connector 19 that is designed to connect the forceps 10 to a source of electrosurgical energy such as an electrosurgical generator (not shown). Proximal shaft connector 19 secures an electrosurgical cable 210 to forceps 10 such that the user may selectively apply electrosurgical energy to the electrically-conductive tissue sealing plates 112 and 122 (see FIGS. 3-4) of jaw members 110 and 120, respectively. More specifically, cable 210 includes one or more wires (not shown) extending therethrough that has sufficient length to extend through one of the shaft members, e.g., shaft member 12b, in order to provide electrical energy to at least one of the sealing plates 112, 122 of jaw members 110, 120, respectively, of end effector assembly 100, e.g., upon activation of activation switch 40b (See FIGS. 1 and 3). Alternatively, forceps 10 may be configured as a battery-powered instrument.

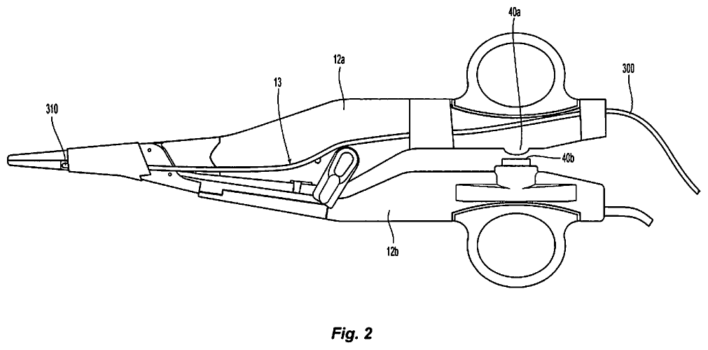

Activation switch 40b is disposed at proximal end 14b of shaft member 12b and extends therefrom towards shaft member 12a. A corresponding surface 40a is defined along shaft member 12a toward proximal end 14a thereof and is configured to actuate activation switch 40b (See FIGS. 1 and 2). More specifically, upon approximation of shaft members 12a, 12b, e.g., when jaw members 110, 120 are moved to the closed position, activation switch 40b is moved into contact with, or in close proximity of surface 40a. Upon further approximation of shaft members 12a, 12b, e.g., upon application of a pre-determined closure force to jaw members 110, 120, activation switch 40b is advanced further into surface 40a to depress activation switch 40b. Activation switch 40b controls the supply of electrosurgical energy to jaw members 110, 120 such that, upon depression of activation switch 40b, electrosurgical energy is supplied to sealing surface 112 and/or sealing surface 122 of jaw members 110, 120, respectively, to seal tissue grasped therebetween. Other more standardized activation switches are also contemplated, e.g., finger switch, toggle switch, foot switch, etc.

Referring now to FIGS. 2 and 3, in conjunction with FIG. 1, forceps 10 may further include a knife assembly (not shown) disposed within one of the shaft members, e.g., shaft member 12a and a knife channel (not shown) defined within one or both of jaw members 110, 120, respectively, to permit reciprocation of a knife (not shown) therethrough. Knife assembly includes a rotatable trigger 144 coupled thereto that is rotatable about a pivot for advancing the knife from a retracted position within shaft member 12a, to an extended position wherein the knife extends into knife channels to divide tissue grasped between jaw members 110, 120. In other words, axial rotation of trigger 144 effects longitudinal translation of knife. Other trigger assemblies are also contemplated.

Each jaw member 110, 120 of end effector assembly 100 may include a jaw frame having a proximal flange extending proximally therefrom that are engagable with one another to permit pivoting of jaw members 110, 120 relative to one another between the open position and the closed position upon movement of shaft members 12a, 12b (FIG. 1) relative to one another between the spaced-apart and approximated or closed positions. Proximal flanges of jaw members 110, 120 also connect jaw members 110, 120 to the respective shaft members 12b, 12a thereof, e.g., via welding.

Jaw members 110, 120 may each further include an insulator (not shown) that is configured to receive an electrically-conductive tissue sealing plate 112, 122, respectively, thereon and that is configured to electrically isolate the tissue sealing plates 112, 122 from the remaining components of the respective jaw members 110, 120 (FIG. 3). In the fully assembled condition, as shown in FIG. 3, tissue sealing plates 112, 122 of jaw members 110, 120 are disposed in opposed relation relative to one another such that, upon movement of jaw members 110, 120 to the closed position, tissue is grasped between tissue sealing plates 112, 122, respectively, thereof. Accordingly, in use, electrosurgical energy may be supplied to one or both of tissue sealing plates 112, 122 and conducted through tissue to seal tissue grasped therebetween and/or knife may be advanced through knife channels of jaw members 110, 120 to cut tissue grasped therebetween.

The simultaneously delivery of electrosurgical energy with saline (sometimes referred to as transcollation technology) is used for hemostatic sealing and coagulation of soft tissue and bone and may be used in a wide variety of surgical procedures, including orthopedic joint replacement, hepatic transection, spinal surgery, orthopedic trauma, and surgical oncology. Utilizing forceps 10 for transcollation-type procedures simultaneously integrates electrosurgical energy and saline to deliver controlled thermal energy to the tissue and allows the tissue temperature to remain at or below 100° C., the boiling point of water. Unlike conventional electrosurgical devices which typically operate at high temperatures, forceps 10 using transcollation technology will not smoke or result in char formation on electrosurgical surfaces when put in contact with tissue “T”. Using transcollation technology and moving forceps 10 in a paint-like or brushing motion will enable surgeons to treat tissue effectively at temperatures at or below 100° C. without producing excess charring. Spot treating bleeding vessels with forceps 10 is also possible. Forceps 10 can seal blood and bile ducts up to 6 mm in diameter and is able to reduce blood loss which makes forceps 10 very useful for these types of procedures. Moreover, forceps 10 has also been found effective for treating cirrhotic livers and destroying potential cancer cells at the margin of resection.

Turning back to FIGS. 1 and 3, and also with reference to FIG. 4, a fluid source “P” is connected to forceps 10. Fluid source “P” may include a bag, a fluid drip or fluid that is pumped to the tissue site. Fluid delivery tubing 300 passes from source “P” to a fluid delivery nozzle 310 disposed at a distal end of forceps 10 proximal to the end effector assembly 100. A channel 13 may be inscribed in forceps 10 to seat fluid line 300 therein and direct fluid line 300 to the distal end of forceps 10. Fluid source “P” may include a peristaltic pump, e.g., a rotary peristaltic pump. Peristaltic pumps may be particularly used, as an electro-mechanical force mechanism, e.g., rollers driven by electric motor, to make contact with the fluid “F”, and force the fluid “F” through the fluid line 300.

Fluid “F” may include liquid saline solution, and even more particularly, normal (0.9% w/v NaCl or physiologic) saline. Although the description herein may make reference to saline as the fluid “F”, other electrically conductive fluids may be used in accordance with the present disclosure. While an electrically conductive fluid having an electrically conductivity similar to normal saline may be particularly useful for many of the treatments described here, fluid “F” may also be an electrically non-conductive fluid depending upon a particular purpose. The use of a non-conductive fluid, while not providing all the advantages of an electrically conductive fluid, still provides certain advantages over the use of a dry electrode including, for example, reduced occurrence of tissue sticking to the forceps 10 and cooling of the electrode and/or tissue. Therefore, it is also within the scope of the present disclosure to include the use of an electrically non-conductive fluid, such as, for example, deionized water.

Before starting a surgical procedure, it may be desirable to prime the forceps 10 with fluid “F” to inhibit power activation without the presence of fluid “F”. A priming switch (not shown) may be used to initiate priming of forceps 10 with fluid “F”. The fluid source “P” may include a fluid flow rate setting display (not shown), e.g., low, medium, and high for example. Selecting the fluid flow rate may be coordinated with the type of tissue treatment, coagulation, cauterizing and/or sealing.

For example, forceps 10 may be configured such that the speed of fluid source “P”, and therefore the throughput of fluid expelled therefrom, is predetermined based on two input variables, the power setting and the fluid flow rate setting. There may be a functional relationship (e.g., linear) of fluid flow rate (cubic centimeters per minute) and the power setting in watts. The relationship may be engineered to inhibit undesirable effects such as tissue desiccation, electrode sticking, smoke production, and char formation, while at the same time not providing a fluid flow rate at a corresponding power setting which is so great as to provide too much electrical dispersion and cooling at the electrode/tissue interface. The fluid from the fluid source “P” may also be provided in a pulsed manner. One or more actuators or dials (not shown) may be employed to selectively vary the pulse of the fluid depending upon a particular purpose. The actuators or dials may be associated with one or both shafts 12a, 12b and/or may be associated with the fluid source “P”.

While not being bound to a particular theory, a more detailed discussion on how the fluid flow rate interacts with the radio frequency power, modes of heat transfer away from the tissue, fractional boiling of the fluid, and various control strategies may be found in U.S. Publication No. 2001/0032002, published Oct. 18, 2001, which is hereby incorporated by reference in its entirety.

As shown in FIG. 4, the power source (not shown) may be connected to the fluid source “P”. For example, the power source may be configured to increase the fluid flow rate (e.g., linearly) with an increasing power setting for each of the above-identified fluid flow rates low, medium and high, respectively. Conversely, the power source may be configured to decrease the fluid flow rate (e.g., linearly) with a decreasing power setting for each of three fluid flow rate settings, respectively. In embodiments, there may be no functional relationship of fluid flow rate versus power setting.

In such an instance, rather than the fluid flow rate being automatically controlled by the power source based on the power setting, the fluid flow rate may be manually controlled, such as by the user of the forceps 10 or another member of the surgical team, with a roller (pinch) clamp or other clamp provided with forceps 10 and configured to act upon and compress the tubing 300 and control flow in a manner known in the art. An example of an electrosurgical unit which does not include a pump, but may be used in conjunction with a manually operated fluid flow control mechanism on forceps 10, includes an electrosurgical unit such as the Force FX™, sold by Covidien a division of Medtronic, Inc.

During use of the forceps 10, a fluid “F” from fluid source “P” is communicated through delivery tubing 300 to the distal end 16a, 16b of the forceps 10 to nozzle 310 disposed proximate end effector assembly 100. Fluid “F”, in addition to providing an electrical coupling between the forceps 10 and tissue “T”, lubricates the surface of tissue “T” and facilitates the movement of electrically conductive plates 112, 122 across surface of tissue “T” for provide the desired tissue effect. During movement of the forceps 10, electrically conductive plates 112, 122 may be typically slid across the surface of tissue “T” in a back and forth painting motion while using fluid “F” as, among other things, a lubricating coating. The amount of or thickness of the fluid “F” between the electrically conductive plates 112, 122 and surface of tissue “T” may be controlled to produce a desired tissue effect.

For example, maintaining the electrically conductive plates 112, 122 within the range of about 0.05 mm to 1.5 mm and providing fluid “F” at a low flow rate will produce a more localized tissue effect, e.g., a low flow rate being defined as a flow rate in the range of about 3 mL to about 15 mL per minute. A selectively removable spacer 400 may be used between the shaft members 12a, 12b (or any other part of the forceps 10) to maintain the electrically conductive plates 112, 122 a specific distance apart within the above identified range.

Nozzle 310 may be selectively extendible and retractable relative to jaw members 110, 120. More particularly, an actuator (not shown) may be included that is associated with one or both shafts 12a, 12b and is configured to selectively extend and retract the nozzle 310 as needed during surgery. The actuator may be electrically or mechanically coupled to the fluid source “P” such that the actuator automatically extends the nozzle 310 when the fluid source “P” is activated to provide fluid and retracts the nozzle 310 when the fluid source “P” is deactivated.

As shown forceps 10 may be used as a bipolar device in which the electrically conductive plates 112, 122 are disposed substantially horizontal relative to the tissue “T”. When forceps 10 is used in this manner, electrically conductive plates 112, 122 are connected to power source and receives bipolar radio frequency energy which forms an alternating current electrical field in tissue “T” located between electrically conductive plates 112, 122 and fluid “F” provided from forceps 10. Fluid “F”, in addition to providing an electrical coupling between the forceps 10 and tissue “T”, lubricates surface of tissue “T” and facilitates the movement of electrically conductive plates 112, 122 across the surface of tissue “T”.

In embodiments, the power source (not shown) or the fluid source “P” may include a sensor “S” (See FIG. 1) that regulates the electrical energy to the tissue “T” such that energy is only delivered when fluid “F” is disposed between electrically conductive plates 112, 122. Moreover, sensor “S” may cooperate with various controls in the power source that regulate energy depending upon the position of the jaw members 110, 120. For example, under typical surgical conditions, if the jaw members 110, 120 were disposed in a spaced-apart position, it would denote a fault condition and energy would not be conducted to the electrically conductive plates 112, 122 (e.g., no tissue between electrically conductive plates 112, 122), however, sensor “S” could override the various controls (or act as part of the overall control system) and energize the electrically conductive plates 112, 122 if fluid “F” were detected between electrically conductive plates 112, 122 or fluid delivery was activated.

The present disclosure also relates to a method of treating tissue and includes orienting a forceps 10 having first and second jaw members 110, 120 including electrically conductive opposing plates 112 and 122 so that an edge of each electrically conductive plates 112, 122 contacts tissue “T”. The first and second jaw members 110, 120 are then opened to create an area “A” therebetween. Fluid “F” is then delivered into the area “A” and the first and second jaw members 110, 120 are then energized. The method includes moving the jaw members 110, 120 across the tissue to create a desired tissue effect. The jaw members 110, 120 may be moved across tissue “T” in a paint-like manner while fluid “F” is being delivered. The fluid “F” may be controlled to regulate the temperature at or below 100° C. to reduce smoke or char formation when the electrically conductive plates 112, 122 contact tissue “T”.

In embodiments, the fluid “F” may be delivered at a rate relative to an amount of energy being provided to the electrically conductive plates 112, 122. The fluid “F” may also be delivered at a rate that is linear to the amount of energy being provided to the electrically conductive plates 112, 122. In yet still other embodiments, a spacer 400 may be provided to maintain the area “A” between the first and second jaw members 110, 120. The method may also include providing one or both jaw members 110, 120 with a spherical distal tip diameter of 5.5 mm +/−1.5 mm evenly split between the two jaw members 110, 120 with rounded edges of minimum radius of 0.5 mm on any part of the jaw members 110, 120 including griping teeth and seal surface to edge transitions. The spherical tip may be used to facilitate liver parenchymal penetration from a depth in the range of about 3.175 mm (0.125 inches) to about 76.2 mm (3 inches).

It is contemplated that the rounded tips of the jaw members 110, 120 may facilitate the device penetrating into parenchyma without possibility of ripping or tearing of blood vessels or bile ducts from possible sharp edges engaging the parenchyma. When dissecting, the rounded configuration of the distal tip will yield a penetration force differential when the jaw members 110, 120 push up against tissue of different types. The force differentials can be used to detect tissue type and prevent unwanted damage to specific types of tissue such as blood vessels, bile ducts and or tumors. The penetration forces for blood vessels, bile ducts and or tumors are approximately three times greater than healthy parenchyma; therefore, the surgeon can detect these areas and avoid damaging tissue by redirecting the penetration of the tool.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A forceps, comprising:
first and second shafts configured to support an end effector assembly at a distal end thereof, the end effector including first and second opposing jaw members each including an electrically conductive plate associated therewith configured to communicate electrosurgical energy therebetween upon selective activation thereof, at least one of the first and second jaw members pivotable relative to the other about a pivot such that the jaw members are selectively movable between an open position wherein the jaw members are spaced relative to one another and a closed position for grasping tissue therebetween; and
a fluid line integrated with at least one of the first and second shafts of the forceps and configured to selectively deliver fluid from a fluid source to the end effector assembly proximate the electrically conductive plates during electrical activation.

2. The forceps according to claim 1, further comprising a sensor configured to sense the presence of fluid between the electrically conductive plates prior to selective activation of electrical energy.

3. The forceps according to claim 1, wherein the fluid is delivered through a nozzle disposed at a proximal end of at least one of the electrically conductive plates.

4. The forceps according to claim 1, wherein a rate of delivery of the fluid relates to an amount of energy delivered to the electrically conductive plates.

5. The forceps according to claim 4, wherein the rate of delivery of the fluid is proportional to the amount of energy delivered to the electrically conductive plates.

6. The forceps according to claim 4, wherein the rate of delivery of the fluid is linearly related to the amount energy delivered to the electrically conductive plates.

7. The forceps according to claim 1, wherein the fluid is electrically conductive.

8. The forceps according to claim 1, wherein the fluid is electrically non-conductive.

9. The forceps according to claim 1 wherein the fluid is saline.

10. The forceps according to claim 1 further comprising a spacer selectively disposed between the first and second shafts and configured to maintain the jaw members in the spaced position during electrical activation.

11. A method of treating tissue, comprising:
orienting a forceps having first and second jaw members including electrically conductive opposing plates so that an edge of each electrically conductive plate contacts tissue;
opening the first and second jaw members to create an area therebetween;
delivering fluid to the area defined between the first and second jaw members;
energizing the electrically conductive plates; and
moving the first and second jaw members across tissue to create a desired tissue effect.

12. The method of treating tissue according to claim 11, further comprising moving the first and second jaw members in a paint brush-like manner across tissue.

13. The method of treating tissue according to claim 11, wherein the fluid is delivered at a rate relative to an amount of energy being provided to the electrically conductive plates.

14. The method of treating tissue according to claim 11, wherein the fluid is delivered at a rate that is linear to an amount of energy being provided to the electrically conductive plates.

15. The method of treating tissue according to claim 11, further comprising providing a spacer to maintain the area between the first and second jaw members.
